(19) United States
(12) Patent Application Publication
(10) Pub. No.: US 2004/0254391 A1
(43) Pub. Date: Dec. 16, 2004

(54) Process for the preparation of nitrile compounds

(76) Inventors: Andrew John Blacker, West Yorkshire (GB); Ian Nicholas Houson, West Yorkshire (GB); Jonathan William Wiffen, West Yorkshire (GB)

Correspondence Address: MORGAN LEWIS & BOCKIUS LLP, 1111 PENNSYLVANIA AVENUE NW, WASHINGTON, DC, 20004, US

(21) Appl. No.: 10/482,381

(22) PCT Filed: Jun. 27, 2002
(86) PCT No.: PCT/GB02/02964

(30) Foreign Application Priority Data
Jul. 3, 2001 (GB) 0116212.2

(51) Int. Cl. C07C 253/16

(52) U.S. Cl. 558/350

(57) ABSTRACT

A process is provided for the preparation of a compound of Formula (1)

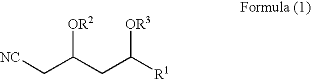

wherein:

R¹ is H, optionally substituted acyl, optionally substituted alkyl, optionally substituted aryl or optionally substituted heteroaryl:

R² and R³ each independently are H or a hydroxy protecting group; comprising the steps:

(a) reacting a compound of Formula (2)

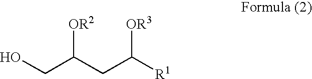

in a solvent in the presence of a base with a compound of formula R⁴SO₂X to give a compound of Formula (3);

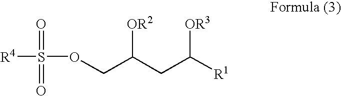

wherein:

R⁴ is an optionally substituted alkyl, optionally substituted aryl or optionally substituted heteroaryl group; and X is halogen: and

(b) reacting the compound of Formula (3) with a cyanide source in the presence of a phase transfer catalyst.

This invention relates to processes for the preparation of aliphatic nitriles substituted in the 3 and 5 positions with hydroxyls or protected hydroxyls.

Aliphatic nitrites substituted in the 3 and 5 positions with protected alcohols are important intermediate in the synthesis of pharmaceuticals. For example (6S-cyanomethyl-2,2-dimethyl-[1,3]dioxan-4R-yl)-acetic acid tert-butyl ester is a key intermediate in the synthesis of Atorvastatin ((2R-trans)-5-(4-fluorophenyl)-2-(1-methylethyl)-N,4-diphenyl]-1-[2-(tetrahydro-4-hydroxy-6-oxo-2H-pyran-2-yl)ethyl]-1H-pyrrole-3-carboxamide (U.S. Pat. Nos. 4,647,576 and 4,681,893) ) the active agent in Lipitor™ which is used as a hypolipidemic and hypocholesterolemic agent.

One method of making an aliphatic nitrile is to convert the corresponding primary alcohol to an active intermediate such as a sulphonyloxy or alkyl halide then cyanylating to yield a nitrile.

The displacement of sulphonyloxy groups by cyanide is well known in the art. However, such displacements can be difficult in complex systems. For example, Sunay, U. and Fraser-Reid, B., Tetrahedron Letters, 27, pages 5335-5338 (1986) were unable to displace sulphonyloxy groups by cyanide in a compound containing a 1,3-dioxane ring. They also noted that the mesyl sulphonyloxy analogue of this compound was unstable on standing.

In U.S. Pat. No. 5,103,024 displacement of a substituted phenyl sulphonyloxy group by cyanide in a system containing a 1,3-dioxane ring was achieved. However, the reaction was extremely slow taking several days. This was confirmed by Brower et al (Tetrahedron Letters 33, 2279-2282) who noted that displacement of mesylate from (6S-methanesulphonyloxymethyl-2,2-dimethyl-[1,3]dioxan-4R-yl)-acetic acid tert-butyl ester or tosylate from (6S-tosylsulphonyloxymethyl-2,2-dimethyl-[1,3]dioxan-4R-yl)-acetic acid tert-butyl ester by cyanide required weeks to achieve significant conversion.

Thus, processes of this type are extremely slow and potentially involve an unstable intermediate both of which potentially limit their commercial applicability.

According to the present invention there is provided a process for the preparation of a compound of Formula (1)

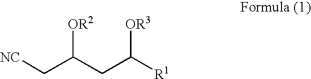

wherein:

R¹ is H, optionally substituted acyl, optionally substituted alkyl, optionally substituted aryl or optionally substituted heteroaryl:

R² and R³ each independently are H or a hydroxy protecting group; comprising the steps:

(a) reacting a compound of Formula (2)

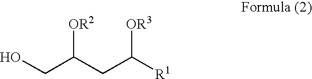

in the presence of a base with a compound of formula R⁴SO₂X to give a compound of Formula (3);

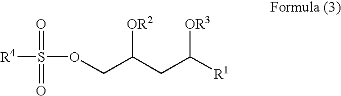

wherein:

R⁴ is an optionally substituted alkyl, optionally substituted aryl or optionally substituted heteroaryl group; and X is halogen: and

(b) reacting the compound of Formula (3) with a cyanide source in the presence of a phase transfer catalyst.

The process for the conversion of a compound of Formula (3) to a compound of Formula (1) forms a second aspect of the present invention. Thus the second aspect of the invention provides a process for the preparation of a compound of Formula (1)

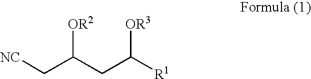

wherein:

R¹ is H, optionally substituted acyl, optionally substituted alkyl, optionally substituted aryl or optionally substituted heteroaryl:

R² and R³ each independently are H or a hydroxy protecting group; which comprises reacting a compound of Formula (3)

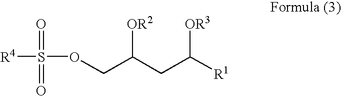

wherein

R⁴ is an optionally substituted alkyl, optionally substituted aryl or optionally substituted heteroaryl group; with a cyanide source in the presence of a phase transfer catalyst.

R¹ in Formulae (1), (2) and (3) is preferably a group of formula —C(═O)—Z wherein Z is optionally substituted alkyl, optionally substituted aryl or optionally substituted heteroaryl group, more preferably optionally substituted C₁₋₁₂alkyl and especially optionally substituted C₁₋₄alkyl.

Preferred optional substituents which may be present on R¹ are optionally substituted alkyl, preferably C₁₋₄-alkyl; optionally substituted alkoxy, preferably C₁₋₄-alkoxy; optionally substituted aryl, preferably phenyl; optionally substituted aryloxy, preferably phenoxy; polyalkylene oxide; carboxy; phosphate; sulpho; nitro; cyano; halo; ureido; —SO₂F; hydroxy; ester, preferably carboxyester; —NR⁵R⁶; —COR⁵; —CONR⁵R⁶; —NHCOR⁵; sulphone; and —SO₂NR⁵R⁶ wherein R⁵ and R⁶ are each independently H, optionally substituted alkyl, especially C₁₋₄-alkyl, or optionally substituted aryl, especially phenyl, or, in the case of —NR⁵R⁶ , —CONR⁵R⁶ and —SO₂NR⁵R⁶, R⁵ and R⁶ together with the nitrogen atom to which they are attached represent an aliphatic or aromatic ring system. Preferred optional substituents which may be present on R⁵ and R⁶ are carboxy; phosphato; sulpho; nitro; cyano; halo; ureido; —SO₂F; hydroxy. R⁵ and R⁶ are often unsubstituted.

R¹ is preferably substituted with an ester or a group capable of forming an ester such as hydroxy or carboxy. Most preferably R¹ has an ester substituent. It is particularly preferred that R¹ is a group of formula —CH₂CO₂R⁷ wherein R⁷ is optionally substituted alkyl, optionally substituted aryl or optionally substituted heteroaryl.

In view of the above preferences a favoured compound of Formula (1) is of Formula (4):

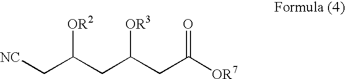

wherein R⁷ is optionally substituted alkyl, optionally substituted aryl or optionally substituted heteroaryl.

It is particularly preferred that R⁷ is optionally substituted alkyl more preferably optionally substituted C₁₋₁₂alkyl and especially optionally substituted C₁₋₄alkyl.

The preferred optional substituents for R⁷ are the same as those listed above for R¹.

In a particularly favoured embodiment R¹ is —CH₂C(═O)OtBu.

Preferably the hydroxy protecting groups, R² and R³ each independently are optionally substituted alkyl, optionally substituted aryl or optionally substituted heteroaryl or R² and R³ together with the oxygen atoms to which they are attached comprise an optionally substituted ring system.

It is preferred that R² and R³ together with the oxygen atoms to which they are attached comprise an optionally substituted ring system. It is particularly preferred that R² and R³ form a 1,3 dioxane ring via the oxygen atoms to which they are attached.

Preferred optional substituents that may independently be present on R², R³, R⁴ and Z are the same as those listed above for R¹.

Thus, a further preferred compound of Formula (1) is of Formula (5).

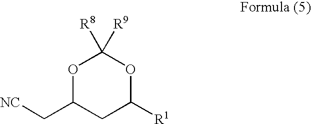

wherein R⁸ and R⁹ are optional substituents

Preferably R⁸ and R⁹ are optionally substituted C₁₋₄alkyl, more preferably methyl.

Preferred optional substituents for R⁸ and R⁹ are as listed above for R¹.

It is especially preferred that R² and R³ together with the oxygen atoms to which they are attached form a 2,2-dimethyl-1,3-dioxane moiety, more especially a 4R,6S-cis-2,2-dimethyl-1,3-dioxane moiety.

Compounds of Formulae (1) to (5) that comprise acid or basic groups on the compound can exist either as a free acid or base or in the form of a salt. Thus, the Formulae shown herein include compounds in both forms.

In view of the above preferences a particularly favoured compound of Formula (1) is of Formula (6):

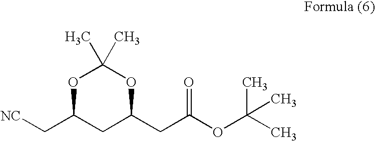

Preferred compounds of Formulae (2) and (3) are selected accordingly.

In step (a) and step (b) it is preferred that that R⁴ is optionally substituted alkyl. It is particularly preferred that R⁴ is C₁₋₄-alkyl or C₁₋₄-alkyl optionally substituted with a halogen, particularly fluorine. R⁴ is most favourably methyl or mono, di or trifluoromethyl.

In step (a) X is preferably chloro.

Step (a) of the process is preferably performed in the presence of any organic solvent or mixture of organic solvents which is unreactive towards the reagents employed. Examples of suitable solvents include halocarbons, especially chlorocarbons such as dichloromethane, chloroform, dichloroethane, chlorobenzene; ethers, particularly C₁₋₆ alkylethers such as t-butyl methyl ether and tetrahydrofuran; and hydrocarbons particularly toluene; and mixtures thereof. Preferably the solvent is dichloromethane, toluene or t-butyl methyl ether. More preferably the solvent is toluene.

Any compatible base may be added to the reaction mixture in step (a). Preferably the base is: an amine, more preferably an alkyl amine; a heteroaromatic base such as pyridine, or an aryl amine; or an inorganic base such as CaO, Na₂CO₃ or K₂CO₃. It is particularly preferred that the base is a trialkylamine especially a tri(C₁₋₄)alkylamine.

Step (a) of the process is preferably performed at a temperature in the range of from −20° C. to 90° C. and more preferably in a range from 5° C. and 50° C. It is especially preferred that step (a) is carried out at ambient temperature such as from 15° C. to 35° C.

Step (a) of the process is advantageously allowed to proceed to at least 90% conversion to a compound of Formula (3).

The reaction time of step (a) of the process of the present invention will depend on a number of factors, for example the reagent concentrations, the relative amounts of reagents and particularly the reaction temperature. Typical reaction times, in addition to the reagent addition times, range from 1 minute to 48 hours, with reaction times of 5 minutes to 20 hours being common.

Preferably the cyanide source is either (i) a compound of formula Y(CN)ₓ where Y is a cation of valency x and x is a positive integer, preferably 1 or 2 or (ii) a complexed cyanide source. The complexed cyanide source may be a cyanohydrin, acyl cyanide, a cyanoformate, a tosyl or other aryl or alkyl cyanide, sulphonyl cyanide, a silyl cyanides such as trimethylsilyl cyanide, or an alkyl transition metal cyanide such as tributyl tin cyanide. More preferably the cyanide source is a compound of formula Y(CN)ₓ as defined above wherein Y is H; ammonium, which herein includes NH₄⁺and ammonium salts of amines; heteroaromatic bases such as pyridine; or an alkali, alkaline earth or transition metal. Most preferably the cyanide source is lithium, sodium, potassium or ammonium cyanide or a quaternary ammonium cyanide salt.

The complexed cyanide source may be a cyanohydrin, acyl cyanide, a cyanoformate, a tosyl or other aryl or alkyl cyanide, sulphonyl cyanide, a silyl cyanide such as trimethylsilyl cyanide, or an alkyl transition metal cyanide such as tributyl tin cyanide.

Preferred phase transfer catalysts are quaternary ammonium compounds; crown ethers; linear and branched ethers such as polyalkylene ethers, preferably alkyl capped polyalkylene ethers including tetraethylene glycol dimethyl ether, polyglycol DME500, polyglycol DME 2000 and tris(dioxa-3,6-heptyl)amine (TDA-1); aryl amines; branched nitrogen based dendrimers; branched oxygen base dendrimers or macrocycles; phosphonium salts; and guanidine or amidine bases such as 1,1,3,3-tetramethylguanidine (TMG) or 1,8-diazabicyclo[5.4.0]undec-7-ene (DBU).

Preferred quaternary ammonium compounds are tetraalkylammonium salts wherein the alkyl groups typically independently comprise from 1 to 18 C atoms and alkyl aryl ammonium compounds e.g. trialkyl aryl ammonium compounds. Preferred anions include hydroxide, sulphate and halide especially chloride and bromide.

Examples of preferred quaternary ammonium compounds include tetramethylammionium chloride, tetraethylammonium bromide, tetraethylammonium hydroxide, tetrapropylammonium bromide, tetrapropylammonium hydroxide, tetrabutylammonium bromide, tetrabutylammonium fluoride, tetrabutylammonium sulphate, tetrabutylammonium iodide, tetrabutylammonium tribromide, benzyltriethylammonium chloride, cetyltrimethylammonium bromide, tetradecyltrimethyl ammonium bromide, tetraethylammonium iodide, tetraheptyl ammonium bromide, tetraheptyl ammonium chloride, tetrahexadecyl ammonium bromide, tetrahexyl ammonium bromide, tetrahexyl ammonium chloride, tetramethyl ammonium hydroxide, tetramethyl ammonium iodide, tetraoctadecyl ammonium bromide, tetrapentyl ammonium bromide, tetrapentyl ammonium chloride, tridocecylmethyl ammonium bromide, tridocecylmethyl ammonium chloride, tridocecylmethyl ammonium iodide, triethylhexyl ammonium bromide, triethylmethyl ammonium bromide, triethylmethyl ammonium chloride, trimethylphenyl ammonium bromide, trimethylphenyl ammonium chloride, trimethylphenyl ammonium iodide, trimethylphenyl ammonium tribromide.

If the phase transfer catalyst is a quaternary amine it may be present as a cyanide salt and so act as both a cyanide source and as a phase transfer catalyst. Examples of such compounds are tetraethyl ammonium cyanide and tetrabutyl ammonium cyanide.

Examples of phosphonium catalysts include but are not limited to tetrabutylphosphonium bromide, tetrabutylphosphonium chloride, tetrabutylphosphonium hydroxide, tetraethylphosphonium bromide, tetraethylphosphonium chloride, tetraoctadecyl phosphonium bromide, tetraphenyl phosphonium bromide, tetraphenyl phosphonium chloride, tetraphenyl phosphonium iodide.

More preferably the phase transfer catalyst is a crown ether, linear crown ether, branched nitrogen based dendrimer, branched oxygen base dendrimer or macrocycle and most preferably a crown ether. The nature of the crown ether selected will vary with the cyanide source used in step (b). In particular it will vary according to the nature of Y. For example when Y is sodium a preferred crown ether is 15-crown-5 and when Y is potassium a preferred crown ether is dicyclohexano-18-crown-6. Other crown ethers which may be used include dibenzo-18-crown-6, dibenzo-21-crown-7, dibenzo-24-crown-8, dibenzo-30-crown-10, dicyclohexano-18-crown-6, 18-crown-6, 21-crown-7, 24-crown-8, 30-crown-10, benzo-18-crown-6, cyclohexyl-18-crown-6.

Mixtures of 2 or more different phase transfer catalysts may be employed if desired.

Step (b) and the second aspect of the invention can be performed in the absence of or presence of any solvent or mixture of solvents that is unreactive towards the reagents employed.

The solvent used in step (b) and the second aspect of the invention preferably comprises water and/or organic solvent or a mixture of organic solvents. Preferred organic solvents are water-miscible organic solvents, water immiscible organic solvents and mixtures thereof.

When the solvent comprises water it may be an aqueous buffer preferably in the pH range of pH 6 to 14 and more preferably in the range pH 8 to 12 and especially pH 9 to 11.

Suitable water-miscible organic solvents include ethers, N,N-dimethylformamide, dimethylsuphoxide, tetrahydrofuran, acetonitrile, methanol and sulpholane.

Suitable water-immiscible organic solvents include toluene, 2,2,4-trimethylpentane, hexane, heptane, octane, cyclohexane, methylcyclohexane, alkanes, branched alkane, alkenes and arynes.

Preferred solvent systems for step (b) and the second aspect of the invention are water; water and starting material oil preferably comprising from 10 to 99% w/w water; or a mixture of acetonitrile and N,N-dimethylformamide preferably comprising from 5 to 80% w/w acetonitrile.

A particularly preferred solvent system for step (b) and the second aspect of the invention comprises an aqueous buffer preferably in the pH range of 9 to 11.

Step (b) and the second aspect of the invention may be carried out in the presence of oxygen though preferably oxygen is omitted and step (b) or the second aspect of the invention is carried out under a nitrogen or inert gas atmosphere.

Step (b) and the second aspect of the invention of the process is preferably performed at a temperature in the range of from −20° C. to 98° C. and more preferably in the range of from 45° C. to 95° C. It is especially preferred that step (b) is carried out at a temperature in the range of from 60° C. to 90° C.

Step (b) and the second aspect of the invention of the process is advantageously allowed to proceed to at least 50% conversion to a compound of Formula (1).

The reaction time of step (a) of the process of the present invention will depend on a number of factors, for example the reagent concentrations, the relative amounts of reagents, the nature of the catalyst and particularly the reaction temperature. Typical reaction times, in addition to the reagent addition times, range from 1 hour to 300 hours, with reaction times of 1 hour to 48 hours being common.

The product of step (a) may be isolated prior to step (b). However, preferably the product of step (a) is used in step (b) without any further processing or purification.

A preferred embodiment of the present process is a process for the preparation of a compound of Formula (6)

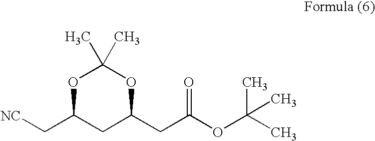

comprising the steps:

(a) reacting a compound of Formula (7);

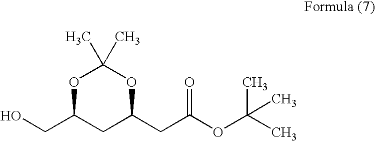

in a solvent in the presence of a base with a compound of formula R⁴SO₂X to give a compound of Formula (8);

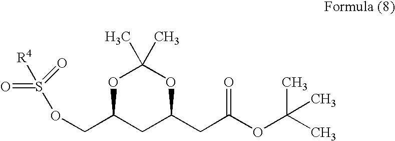

wherein:

R⁴ is an optionally substituted alkyl, optionally substituted aryl or optionally substituted heteroaryl group; and X is halogen:

(b) reacting a compound of Formula (8) with either a compound of formula YCN, wherein Y is H, ammonia, tertiary amine, heteroaromatic base, aryl amine or an alkali, alkaline earth or transition metal, or with a complexed cyanide source in the presence of a phase transfer catalyst.

A more preferred embodiment of the present process is a process for the preparation of a compound of Formula (6) comprising the steps:

(a) reacting a compound of Formula (7) in toluene in the presence of triethylamine with methanesulphonyl chloride to give a compound of Formula (9);

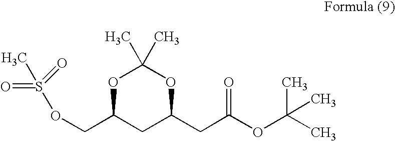

(b) reacting a compound of Formula (9) with a compound of formula YCN, wherein Y is H, ammonia, sodium or potassium, in the presence of a crown ether.

The compounds of Formula (1) to (9) may exist in tautomeric forms and salts other than those shown in this specification. These tautomers and salts are included within the scope of the present invention.

The invention is further illustrated below wherein all parts and percentages are by weight unless otherwise stated.

### COMPARATIVE EXAMPLE 1

### Preparation of (6S-cyanomethyl-2.2-dimethyl-[1.3]dioxan-4R-yl)-acetic acid tert-butyl ester

Step (a)

### Preparation of (6S-Methanesulphonyloxymethyl-2.2-dimethyl-[1.3]dioxan-4R-yl)-acetic acid tert-butyl ester.

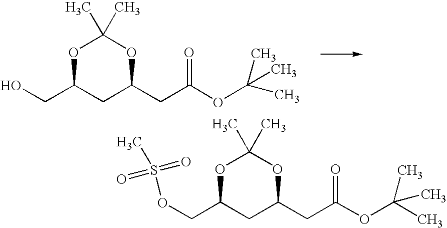

| Reactant | Wt/Vol | | Mol.Wt | Mol | Mol ratio |
|---|---|---|---|---|---|
| (6S-Hydroxymethyl-2,2- | 93.0 | g | 260.33 | 0.357 | 1.0 |
| dimethyl-[1,3]dioxan-4R-yl)- | | | | | |
| acetic acid tert-butyl ester | | | | | |
| Methanesulphonyl chloride | 55.3 | mL | 114.55 | 0.714 | 2.0 |
| Triethylamine | 149 | mL | 101 | 1.071 | 3.0 |
| Dichloromethane | 1.5 | L | 85 | 13.3 | 37.3 |
| Water | 3.6 | L | 18 | 200 | 560.2 |
| Anhydrous Sodium Sulphate | 50 | g | 142 | 0.35 | 1 |

An oven dried 3 L 3-necked flask was fitted with an overhead stirrer and thermometer and placed under an inert nitrogen atmosphere by back filling three times with nitrogen. Methanesulphonyl chloride (55.3 mL) as a dichloromethane solution (in 1 L) was charged to the flask and cooled to 0° C. using a brine/ice bath with stirring. A solution of (6S-hydroxymethyl-2,2-dimethyl-[1,3]dioxan-4R-yl)-acetic acid tert-butyl ester (93 g) in dichloromethane (500 mL) was added drop-wise over 1 hour followed by a solution of triethylamine (149 mL) in dichloromethane (500 mL) over 30 minutes. The reaction mass was left at 0° C. for 2 hours when the cooling was removed and the reaction mass stirred for 24 hours at ambient temperature. The resulting orange solution was washed with water (3×1.2 L) and dried over anhydrous sodium sulphate. The solution was concentrated in vacuo to afford a dark brown viscous oil which solidified on standing in 97% yield. The material was used without further purification in step (b) the cyanation step. The product of step (a) can be further purified as a white solid by recrystallising from hexane.

Step (b)

### Preparation of the Title Product

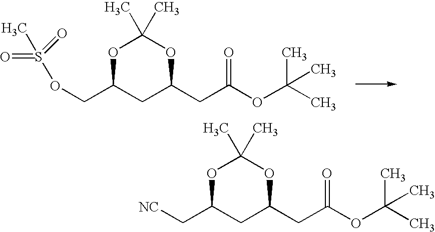

An oven dried 3 necked 1 L flask fitted with an overhead stirrer and thermometer was charged with the product of step (a) ((6S-methanesulphonyloxymethyl-2,2-dimethyl-[1,3]dioxan-4R-yl)-acetic acid tert-butyl ester) (33.4g) and sodium cyanide (24.3 g) before being placed under a nitrogen atmosphere by back filling with nitrogen three times. Dimethylsulphoxide (500 mL) was added and the reaction mass was warmed, with stirring, to 45° C. for 192 hours. The reaction was quenched into water (1000 mL) before being extracted with diethyl ether (3×400 mL). The diethyl ether extracts were combined, washed with water (3×400 mL) and then brine (2.5M, 400 mL) before being dried over anhydrous sodium sulphate. The solvent was removed in vacuo and the resulting residues recrystallised from hexane to afford the desired compound as an off-white powder in 51% isolated yield.

### EXAMPLE 1

### Preparation of (6S-cyanomethyl-2.2-dimethyl-[1.3]dioxan-4R-yl)-acetic acid tert-butyl ester

| | | % | 100% | | mol |
|---|---|---|---|---|---|
| Reactant | Act Wt | strength | wt | moles | ratio |
| (6S-Hydroxymethyl-2,2- | 100 g | 57.7 | 57.7 g | 0.222 | 1.000 |
| dimethyl-[1,3]dioxan-4R- | | | | | |
| yl)-acetic acid tert-butyl | | | | | |
| ester in toluene | | | | | |
| Methanesulphonyl chloride | 28.5 g | 99.5 | 28.3 g | 0.247 | 1.11 |
| | 19.28 ml | | | | |
| Triethylamine | 34.45 g | 99% | 34.1 g | 0.337 | 1.52 |
| Toluene | 186 g | | | | |
| Water 1 | 300 | | | | |
| 5% Sodium Bicarbonate | 500 ml | 5 | 500 ml | | |
| solution | | | | | |
| 10% Brine | 500 ml | | 500 ml | | |
| Water 2 | 46.6 | | | | |

(6S-Hydroxymethyl-2,2-dimethyl-[1,3]dioxan-4R-yl)-acetic acid tert-butyl ester in toluene (100 g) was charged to a 1 L split necked reaction flask under a nitrogen blanket. Anhydrous toluene (186 g) and triethylamine (34.45 g) were added and the temperature was kept below 30° C. Methanesulphonyl chloride (28.5 g) was then added dropwise to the solution over 1 hour and the reaction was cooled to maintain the temperature at 22±6° C. The reaction mixture was then held at 22±6° C. for 1 hour. Water (300 ml) was then added and the resultant mixture was stirred for 1.5 hours. The organic phase was taken and washed with 5% sodium bicarbonate solution (500 ml), twice with water (2×250 ml) and then with 10% brine (500 ml). Solvent was removed from the reaction mixture using a rotary evaporator at below 35° C. The product was obtained in 92-95% yield (69.9 to 72.2 g).

Step (b)

### Preparation of (6S-cyanomethyl-2.2-dimethyl-[1,3]dioxan-4R-yl)-acetic acid tert-butyl ester

The product of step (a) ((6S-methanesulphonyloxymethyl-2,2-dimethyl-[1,3]dioxan-4R-yl)-acetic acid tert-butyl ester) was used to prepare a 60% slurry in water (116.7 g of slurry). Potassium cyanide (18.1 g) and dicyclohexano-18-C-6 crown ether (10.02 g) were charged to this aqueous slurry at 35° C. The reaction mixture was heated to 80° C. and held at this temperature until the reaction was complete (24hrs) as judged by GLC. The reaction yield was 80%. The product was dissolved in toluene (57 g) and the two phases were separated. The toluene layer was filtered sequentially through the two Fullers Earth columns pre-wetted with toluene (26 mm×42 mm) to remove the crown ether and decolourize the product. The toluene was removed by distillation and exchanged for hexane (133.7 g). The product was then crystallised from hexane (20% w/w) by dissolving at 55° C. and cooling over 2 hours to −10° C. The white to pale yellow crystals were filtered and displacement washed with cold hexane to afford 33.4 g product at 60% yield.

### EXAMPLE 2

Step (a)

### Preparation of (6S-Methanesulphonyloxymethyl-2.2-dimethyl-[1,3]dioxan-4R-yl)-acetic acid tert-butyl ester

This was carried out as in Example 1 step (a).

Step (b)

### Preparation of (6S-cyanomethyl-2.2-dimethyl-[1,3]dioxan-4R-yl)-acetic acid tert-butyl ester

Potassium cyanide (20.8 g), dicyclohexano-crown ether 18-c-6 (16.6 g), the product of step (a) (70 g) and 0.1M borate buffer, pH 10 (46.5 g) were added to a reaction vessel at 35° C. The reaction mixture was heated to 80° C. and held at this temperature for 35 hours. Water (100 g) was then added and the mixture was stirred and then allowed to settle before removing 100 ml of the lower phase. The temperature of the reaction mixture was adjusted to 35° C. and potassium cyanide (20.8 g), crown ether 18-c-6 (16.6 g) and water (46.5 g) were added. The reaction mixture was then reheated to 80° C. and held at this temperature for 30 hrs. The product was dissolved in toluene (100 ml) and the two phases were separated. The toluene phase was then washed with water (4×50 ml) to remove residual cyanide. The product was further purified by passing through a alumina column (3 cm×12 cm). Toluene was then removed by distillation (<40° C.) and exchanged for heptane (133.7 g). The product was crystallised from heptane (15% w/w) by dissolving at 55° C. followed by cooling over 2 hours to −10° C. The white to pale yellow crystals were filtered and the resultant slurry was washed with ice cold hexane and dried to yield 27.8 g of product.

1. A process for the preparation of a compound of Formula (1) 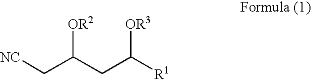
wherein:
R¹ is H, optionally substituted acyl, optionally substituted alkyl, optionally substituted aryl or optionally substituted heteroaryl;
R² and R³ each independently are H or a hydroxy protecting group;
comprising the steps:
(a) reacting a compound of Formula (2) 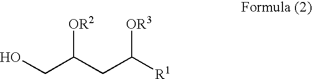
in a solvent in the presence of a base with a compound of formula R⁴SO₂X to give a compound of Formula (3); 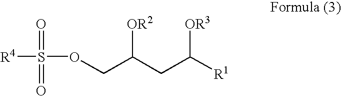
wherein:
R⁴ is an optionally substituted alkyl, optionally substituted aryl or optionally substituted heteroaryl group; and X is halogen; and
(b) reacting the compound of Formula (3) with a cyanide source in the presence of a phase transfer catalyst.

2. A process according to claim 1 wherein the compound of Formula (1) is of Formula (4): 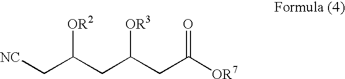
wherein R⁷ is optionally substituted alkyl, optionally substituted aryl or optionally substituted heteroaryl.

3. A process according to claim 1 wherein the compound of Formula (1) is of Formula (5)

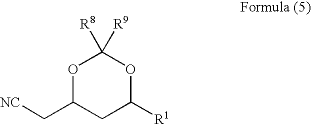

4. A process according to claim 1 for the preparation of a compound of Formula (6) 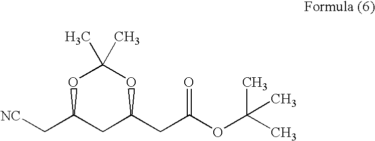
comprising the steps:
(a) reacting a compound of Formula (7); 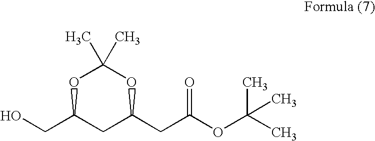
in a solvent in the presence of a base with a compound of formula R⁴SO₂X to give a compound of Formula (8); 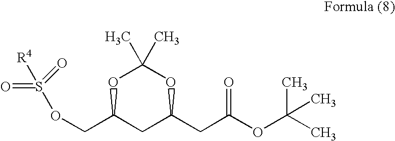
wherein:
R⁴ is an optionally substituted alkyl, optionally substituted aryl or optionally substituted heteroaryl group; and X is halogen:
(b) reacting a compound of Formula (8) with a cyanide source in the presence of a phase transfer catalyst.

5. A process according to claim 1 wherein R⁴ is methyl or mono, di or trifluoromethyl.

6. A process according to claim 1 wherein in step (a) the solvent is dichloromethane, toluene or t-butyl methyl ether.

7. A process according to claim 1 wherein the cyanide source is either (i) a compound of formula Y(CN)ₓ where Y is a cation of valency x and x is a positive integer, preferably 1 or 2 or (ii) a complexed cyanide source.

8. A process according to claim 1 wherein the phase transfer catalyst is a crown ether.

9. A process according to claim 1 for the preparation of a compound of Formula (6) comprising the steps:
(a) reacting a compound of Formula (7) in toluene in the presence of triethylamine with methanesulphonyl chloride to give a compound of Formula (9); 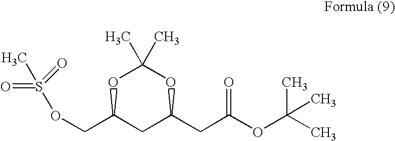
(b) reacting a compound of Formula (9) with a compound of formula YCN, wherein Y is H, ammonia, lithium, sodium or potassium, in the presence of a crown ether.

10. A process for the preparation of a compound of Formula (1) 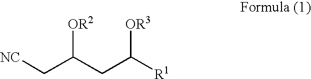
wherein:
R¹ is H, optionally substituted acyl, optionally substituted alkyl, optionally substituted aryl or optionally substituted heteroaryl:
R² and R³ each independently are H or a hydroxy protecting group;
which comprises reacting a compound of Formula (3) 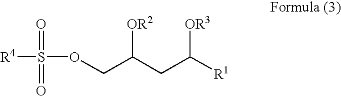
wherein
R⁴ is an optionally substituted alkyl, optionally substituted aryl or optionally substituted heteroaryl group;
with a cyanide source in the presence of a phase transfer catalyst.
